(19) United States
(12) Patent Application Publication
(10) Pub. No.: US 2006/0241638 A1
(43) Pub. Date: Oct. 26, 2006

(54) Anatomical landmark guide

(75) Inventors: James E. Grimm, Winona Lake, IN (US); Shawn E. McGinley, Fort Wayne, IN (US)

Correspondence Address: ZIMMER TECHNOLOGY - ROBERTS, P.O. BOX 1268, ALEDO, TX, 76008, US

(73) Assignee: Zimmer Technology, Inc.

(21) Appl. No.: 11/102,338

(22) Filed: Apr. 8, 2005

(51) Int. Cl. A61B 17/58

(52) U.S. Cl. 606/88

(57) ABSTRACT

An apparatus for indicating a plurality of anatomical landmark locations includes a tracking device and a guide member extending from the tracking device. The guide member is configured to space the tracking device away from a first and a second one of the plurality of anatomical landmark locations, and concurrently pivotally position the tracking device based at least in part on a third one of the plurality of anatomical landmark locations. A method for facilitating acquisitions of locations of anatomical landmarks by a computer-assisted knee arthroplasty system includes concurrently abutting a guide member against at least three of the landmarks, and indicating a location of the guide member to the system concurrently with abutting the guide member against the at least three of the landmarks.

### FIELD OF THE INVENTION

The present invention relates generally to the field of orthopaedics, and, more particularly, to an apparatus and method for indicating anatomical landmark locations.

### BACKGROUND

Total joint arthroplasty (“joint replacement”) is the surgical replacement of a joint with a prosthesis. A traditional knee prosthesis has three main components: a femoral implant, a tibial implant, and a tibio-femoral insert. In general, the femoral implant is designed to replace the distal femoral condyles. The femoral implant is typically made from metal. It typically includes medial and lateral rounded surfaces for emulating the medial and lateral condyles, respectively, and an area between the rounded surfaces for emulating the patella sulcus/trochlear region of the distal femur. In general, the tibial implant is designed to support and align the tibio-femoral insert. The tibial implant is also typically made from metal. It typically includes a substantially planar tray or plate portion (“tibial plate”) for supporting the insert, and an elongated stem extending away from the tibial plate for anchoring the tibial implant in the medullary canal of the proximal tibia. In general, the tibio-femoral insert is designed to replace the tibial plateau and the meniscus of the knee. It is typically somewhat disk-shaped, and typically includes one or more substantially planar surfaces for bearing on the tibial plate and one or more generally concave surfaces for bearing against the femoral implant. The insert is typically made of a strong, smooth, low-wearing plastic.

In a traditional total knee arthroplasty (“knee replacement”), the surgeon typically makes an anterior incision spanning over the distal femur, the knee, and the proximal tibia; separates the distal femur and proximal tibia from the surrounding tissues; and hyperflexes, distally extends, and/or otherwise distracts the proximal tibia from the distal femur. Next, the surgeon typically secures a tibial resection guide to the proximal tibia. A resection guide is a jig or template configured to provide a desired cutting angle for a saw blade or other resection tool. Traditional resection guides are used somewhat similarly to the manner in which a carpenter uses a miter box to achieve a desired angle for cutting wood. The surgeon uses the tibial resection guide to position a saw blade or other suitable resection tool and cuts off the tibial plateau (i.e., the upper end of the tibia which forms the lower part of the knee joint). This prepares the proximal tibia to receive the tibial implant (which will form an artificial tibial plateau). To determine the longitudinal axis of the femur, the surgeon inserts an intramedullary (“IM”) rod through a hole near the center of the joint surface of the distal femur and into the medullary canal that extends longitudinally inside the femur. Then, the surgeon aligns one or more femoral resection guides relative to the longitudinal axis of the femur and/or relative to the angle of the artificial tibial plateau, and guides one or more saw blades or other suitable resection tools with the femoral resection guides to cut the distal femur into a proper size and shape for receiving the femoral implant. Next, the surgeon applies cement to the distal femur and/or to the proximal tibia to help hold the femoral implant and/or tibial implant, respectively, in place; the surgeon drives the femoral implant onto the cut surface of the distal femur; and the surgeon drives the stem of the tibial implant generally longitudinally into the intramedullary canal of the proximal tibia. Finally, the surgeon attaches the tibio-femoral insert to the tibial plate and closes the surgical site.

Prosthetic knees are designed to mimic the healthy operation of the natural knees that they replace. A healthy, natural knee is not merely a simple hinged joint that bends backward (flexion). It also has a rotary motion that locks the femoral condyles into the tibial plateau on straightening (extension) of the leg. On extension of the knee, the ligaments become tight and convert the knee into a rigid locked structure. The knee unlocks on flexion, allowing an increased range of motion as the lower leg swings backward. In operation of a conventional knee prosthesis, the lower surface of the femoral implant glides on the upper surface of the tibio-femoral insert (which normally stays sandwiched between the femoral implant and the tibial implant).

However, prosthetic knees may develop complications if the distal femur is not resected properly during the knee replacement (i.e., if the surgeon does not properly size and shape the distal femur for receiving the femoral implant or cut the distal femur at the proper angles relative to the artificial tibial plateau and the longitudinal axis of the femur). Such complications can include increased wear of the plastic surfaces of the prosthesis; bending, cracking or fracture of the bones; dislocation, excessive rotation or loss of motion of the prosthesis; and/or angular deformity of the joint. Naturally, proper resection depends largely on proper alignment of the various resection guides during the knee replacement.

Computerized systems have been developed for facilitating alignment of resection tools and other surgical instruments through modeling and dynamically tracking reference angles, axes, and anatomical landmarks relative to desired results. Such systems typically employ a plurality of position indicators (“trackers” or “tracking devices”) that may be placed on and/or attached to various body parts and/or surgical instruments, sensing equipment for detecting tracker locations, and computers/software for directing instrument alignments. Some such systems have been optically based, including light emitting tracking devices and camera sensors. Some such systems have been electro-magnetically (“EM”) based, including EM reactive tracking devices and EM field generating and sensing equipment. Various other technological bases have also been developed. For example, U.S. Pat. No. 6,205,411 to DiGioia, III et al., entitled “COMPUTER-ASSISTED SURGERY PLANNER AND INTRA-OPERATIVE GUIDANCE SYSTEM,” which is incorporated herein by reference, and U.S. Pat. No. 6,685,711 to Axelson, Jr. et al., entitled “APPARATUS USED IN PERFORMING FEMORAL AND TIBIAL RESECTION IN KNEE SURGERY,” which is also incorporated herein by reference, disclose exemplary computer-assisted systems.

Devices for quickly and reliably indicating locations of anatomical landmarks such as distal femoral condyles are desirable for facilitation of computer-assisted knee replacements.

### SUMMARY OF THE INVENTION

The present invention provides an apparatus for indicating a plurality of anatomical landmark locations. The apparatus includes a tracking device and a guide member extending from the tracking device. The guide member is configured to space the tracking device away from a first and a second one of the plurality of anatomical landmark locations, and concurrently pivotally position the tracking device based at least in part on a third one of the plurality of anatomical landmark locations.

The present invention provides an apparatus for indicating a plurality of anatomical landmark locations. The apparatus includes position indicating means, spacing means, coupled to the indicating means, for spacing the indicating means away from a first and second one of the plurality of anatomical landmark locations, and pivoting means, integrated with the spacing means, for pivotally positioning the position indicating means based at least in part on a third one of the plurality of anatomical landmark locations.

The present invention provides a method for facilitating acquisitions of locations of anatomical landmarks by a computer-assisted knee arthroplasty system. The method includes the steps of concurrently abutting a guide member against at least three of the landmarks, and indicating a location of the guide member to the system concurrently with the step of abutting the guide member against the at least three of the landmarks.

The above-noted features and advantages of the present invention, as well as additional features and advantages, will be readily apparent to those skilled in the art upon reference to the following detailed description and the accompanying drawings.

### BRIEF DESCRIPTION OF THE DRAWINGS

FIG. 1 shows a perspective view of an exemplary anatomical landmark guide apparatus according to the present invention;

FIG. 2 shows a plan view of the apparatus of FIG. 1 (facing the planar surface formed by the arms of the exemplary guide member);

FIG. 3 shows exemplary operational abutment of the apparatus of FIG. 1 against an exemplary medial distal femoral condyle, an exemplary lateral distal femoral condyle, an exemplary medial posterior femoral condyle, and an exemplary lateral posterior femoral condyle; and

FIG. 4 shows a spatial model of exemplary operations of the apparatus of FIG. 1.

### DETAILED DESCRIPTION OF EXEMPLARY EMBODIMENT(S)

Like reference numerals refer to like parts throughout the following description and the accompanying drawings. Additionally, as used herein, the terms “medial,” “medially,” and the like mean pertaining to the middle, in or toward the middle, and/or nearer to the middle of the body when standing upright. Conversely, the terms “lateral,” “laterally,” and the like are used herein as opposed to medial. For example, the medial side of the knee is the side closest to the other knee and the closest sides of the knees are medially facing, whereas the lateral side of the knee is the outside of the knee and is laterally facing. Further, as used herein the term “superior” means closer to the top of the head and/or farther from the bottom of the feet when standing upright. Conversely, the term “inferior” is used herein as opposed to superior. For example, the heart is superior to the stomach and the superior surface of the tongue rests against the palate, whereas the stomach is inferior to the heart and the palate faces inferiorly toward the tongue. Additionally, as used herein the terms “anterior,” “anteriorly,” and the like mean nearer the front or facing away from the front of the body when standing upright, as opposed to “posterior,” “posteriorly,” and the like, which mean nearer the back or facing away from the back of the body.

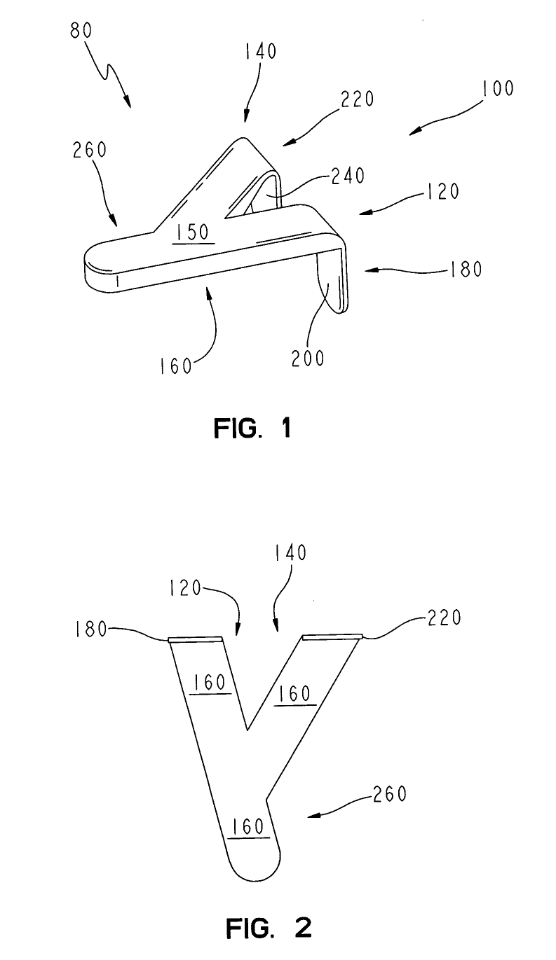

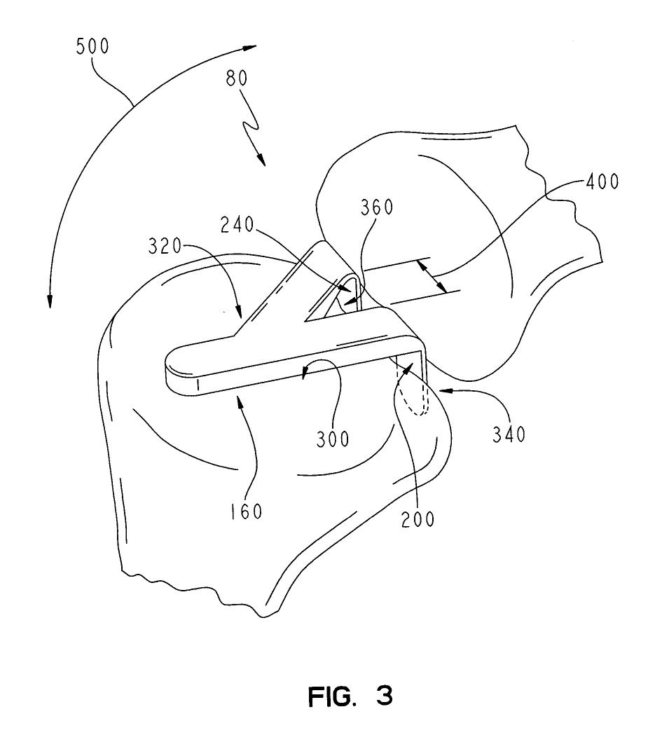

FIG. 1 shows a perspective view of an exemplary anatomical landmark guide apparatus 80 according to the present invention. Exemplary apparatus 80 includes 20 an exemplary guide member 100. In the exemplary embodiment, member 100 is made from one or more biocompatible plastics. In alternative embodiments, member 100 may be made from any other suitable material(s). Member 100 includes an arm 120 and an arm 140 forkedly extending from arm 120. Arm 120 and arm 140 form a substantially planar surface 150 and form a generally inversely facing planar surface 160 (see also FIG. 2). Among other things, surface 160 is configured to slide against distal femoral condyles (see also FIG. 3). Member 100 further includes a tab 180 extending generally perpendicularly from arm 120. Tab 180 forms a planar first surface 200 that is perpendicular to surface 160 (see also FIG. 3). Among other things, surface 200 is configured to slide against a medial posterior femoral condyle. Member 100 further includes a tab 220 extending generally perpendicularly from arm 140. Tab 220 forms a planar second surface 240 that is both perpendicular to surface 160 and coplanar to surface 200 (see also FIG. 3). Among other things, surface 240 is configured to slide against a lateral posterior femoral condyle. Member 100 also includes a butt end portion 260 extending from the convergence of arm 120 and arm 140.

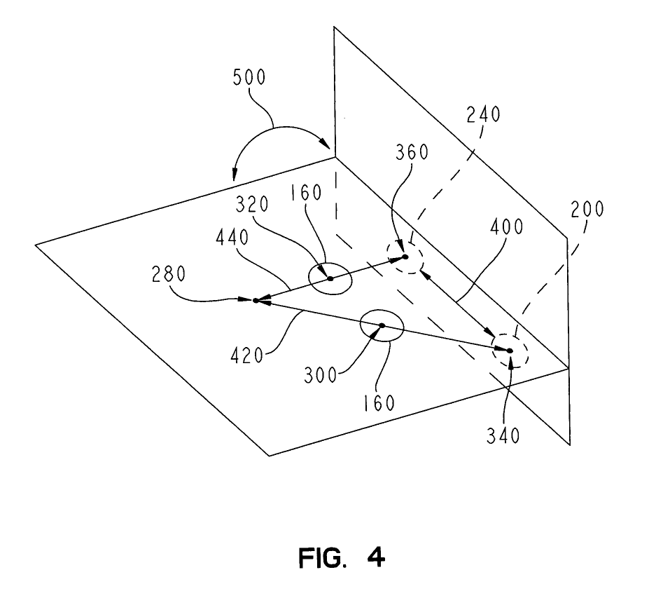

Apparatus 80 further includes a conventional tracking device 280 (see the spatial model of FIG. 4) enclosed within portion 260 of member 100. In the exemplary embodiment, tracking device 280 is an electromagnetic (“EM”) coil type position indicator as known. In alternative embodiments, tracking device 280 may be an optical, radio frequency, and/or gyroscopic position indicator, or any other suitable type of known position indicator.

FIG. 2 shows a plan view of apparatus 80 (facing surface 160). Arm 120, arm 140, surface 160, tab 180, tab 220, and portion 260, among other things, are all at least partially discernable in FIG. 2.

FIG. 3 shows exemplary operational abutment of apparatus 80 against an exemplary medial distal femoral condyle 300, an exemplary lateral distal femoral condyle 320, an exemplary medial posterior femoral condyle 340, and an exemplary lateral posterior femoral condyle 360; and FIG. 4 shows a spatial model of exemplary operations of apparatus 80. In operation of apparatus 80, a surgeon or other operator abuts surface 160 against condyle 300 and concurrently abuts surface 160 against condyle 320. Further, also concurrently, the operator abuts surface 200 against condyle 340 and concurrently abuts surface 240 against condyle 360. The forked extension of arm 140 from arm 120 provides, among other things, a gap 400 between tab 180 and tab 220. Gap 400 facilitates avoidance of the patella (not shown) during procedures in which the patella is not everted. Member 100 spaces tracking device 280 apart from condyle 340 by a predetermined distance 420 and concurrently spaces tracking device 280 apart from condyle 360 by a predetermined distance 440.

In a known manner, an external computer-assisted knee arthroplasty system (not shown) reads an output signal from tracking device 280 and determines a location and pivotal orientation of tracking device 280 (e.g., Cartesian coordinates along predetermined orthogonal “x,” “y,” and “z” reference axes; and pitch, roll, and yaw type angular coordinates relative to each respective axis). The femoral mechanical axis (not shown) is predetermined in a known manner and designated to be one of the reference axes.

While maintaining the abutment of surface 160 against condyle 300 and condyle 320, the abutment of surface 200 against condyle 340, and the abutment of surface 240 against condyle 360, the operator rocks and/or slides surface 160 on condyle 300 and condyle 320, concurrently rocks and/or slides surface 200 on condyle 340, and concurrently rocks and/or slides surface 240 on condyle 360 as necessary (moving apparatus 80 to and/or fro, generally as indicated by directional lines 500) until dynamic feedback from the external system indicates that the operator has moved tracking device 280 into a zero degrees of flexion orientation relative to the mechanical axis of the femur or into any other suitable user-defined orientation. Member 100 provides a resulting pivotal orientation of tracking device 280 (similar to a rotational angle of a knee joint) corresponding to the relative anterior-posterior locations of condyle 340 and condyle 360. Additionally, member 100 provides a resulting pivotal orientation of tracking device 280 (similar to a varus/valgus angle of a knee joint) corresponding to the relative proximal-distal locations of condyle 300 and condyle 320.

Upon achieving the zero flexion or other user-defined orientation, the operator causes the external system to capture the resulting free space location and pivotal orientation of tracking device 280. It should be appreciated that the external system may suitably calculate the relative locations of condyle 300, condyle 340, and condyle 360 based on the known zero flexion angle, distance 420, distance 440, and the remaining tracking device location and orientation data. The external system may suitably incorporate these locations into a computer model of the distal femur for facilitation of a knee replacement as known in the art.

The foregoing description of the invention is illustrative only, and is not intended to limit the scope of the invention to the precise terms set forth. Further, although the invention has been described in detail with reference to certain illustrative embodiments, variations and modifications exist within the scope and spirit of the invention as described and defined in the following claims.

What is claimed is:

1. An apparatus for indicating a plurality of anatomical landmark locations, the apparatus comprising:
a tracking device; and
a guide member extending from the tracking device and configured to space the tracking device away from a first and a second one of the plurality of anatomical landmark locations, and concurrently pivotally position the tracking device based at least in part on a third one of the plurality of anatomical landmark locations.

2. The apparatus of claim 1, wherein the guide member is further configured to position the tracking device in a first pivotal orientation based on the first and second anatomical landmark locations and, and to concurrently position the tracking device in a second pivotal orientation based on the third and fourth one of the plurality of anatomical landmark locations.

3. The apparatus of claim 2, wherein the guide member includes a planar first surface, a planar second surface gapped apart from the first surface, and a planar third surface perpendicular to at least one of the first surface and the second surface.

4. The apparatus of claim 3, wherein the second surface is coplanar to the first surface.

5. The apparatus of claim 1, wherein the tracking device is enclosed within the guide member.

6. An apparatus for indicating a plurality of anatomical landmark locations, the apparatus comprising:
position indicating means;
spacing means, coupled to the indicating means, for spacing the indicating means away from a first and second one of the plurality of anatomical landmark locations; and
pivoting means, integrated with the spacing means, for pivotally positioning the position indicating means based at least in part on a third one of the plurality of anatomical landmark locations.

7. The apparatus of claim 6, wherein the position indicating means is enclosed within the at least one of the spacing means and the pivotally positioning means.

8. A method for facilitating acquisitions of locations of anatomical landmarks by a computer-assisted knee arthroplasty system, the method comprising the steps of:
concurrently abutting a guide member against at least three of the landmarks; and
indicating a location of the guide member to the system concurrently with the step of abutting the guide member against the at least three of the landmarks.

9. The method of claim 8, further comprising the step of aligning the guide member pursuant to feedback from the system.

10. The method of claim 8, further comprising the step of pivotally positioning a tracking device based at least in part on one of the at least three of the landmarks.

11. The method of claim 8, wherein the step of concurrently abutting the guide member against at least three of the landmarks includes concurrently abutting the guide member against at least two distal femoral condyles.

12. The method of claim 11, further comprising the step of pivotally positioning a tracking device based at least in part on one of the two distal femoral condyles.

13. The method of claim 8, wherein the step of concurrently abutting the guide member against at least three of the landmarks includes concurrently abutting the guide member against exactly three of the landmarks.

14. The method of claim 13, wherein concurrently abutting the guide member against exactly three of the landmarks includes concurrently abutting the guide member against at least two distal femoral condyles.

15. The method of claim 14, further comprising the step of pivotally positioning a tracking device based at least in part on one of the two distal femoral condyles.

16. The method of claim 15, further comprising the step of aligning the guide member pursuant to feedback from the system.
